(19) United States
(12) Patent Application Publication
(10) Pub. No.: US 2011/0040363 A1
(43) Pub. Date: Feb. 17, 2011

(54) ELECTRODE LAYOUT METHOD OF HEART TREATMENT APPARATUS

(75) Inventor: Takeshi MORI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/540,736

(22) Filed: Aug. 13, 2009

(51) Int. Cl. A61N 1/05

(52) U.S. Cl. 607/119

(57) ABSTRACT

The purpose is to shorten the operation time required for implanting a heart treatment apparatus into a body by simplifying the lead setting while allowing both heart stimulation and vagus nerve stimulation. There is provided an electrode layout method of a heart treatment apparatus, comprising: upon installation of the heart treatment apparatus comprising an apparatus main body and a lead extending from the apparatus main body, arranging at least one electrode out of a plurality of electrodes provided on the lead in the superior vena cava.

### BACKGROUND OF THE INVENTION

1. Field of the Invention

The present invention relates to an electrode layout method of a heart treatment apparatus.

2. Description of Related Art

Conventionally, there has been known an implantable heart treatment apparatus which increases the heart rate by stimulating the heart when bradycardia occurs, and decreases the heart rate by stimulating the vagus nerve when tachycardia or fibrillation occurs (for example, see Japanese Unexamined Patent Application, Publication No. 2004-173790). In the Japanese Unexamined Patent Application, Publication No. 2004-173790, a heart-stimulating electrode is arranged in a cardiac muscle or an atrium while a nerve-stimulating electrode is arranged by coiling around a vagus nerve in the neck.

### BRIEF SUMMARY OF THE INVENTION

The present invention takes such a situation into consideration with an object of providing an electrode layout method of a heart treatment apparatus, which can shorten the operation time required for implanting the heart treatment apparatus into a body by simplifying the lead setting while allowing both heart stimulation and vagus nerve stimulation.

In order to achieve the above object, the present invention provides the following solution.

The present invention provides an electrode layout method of a heart treatment apparatus, comprising: upon installation of the heart treatment apparatus comprising an apparatus main body and a lead extending from the apparatus main body, arranging at least one electrode out of a plurality of electrodes provided on the lead in the superior vena cava.

### BRIEF DESCRIPTION OF THE SEVERAL VIEWS OF THE DRAWINGS

FIG. 1 illustrates an electrode layout method of a heart treatment apparatus according to one embodiment of the present invention.

FIG. 2 illustrates an example of a method for fixing a venous electrode and a heart electrode.

FIG. 3 illustrates a modified example of the electrode layout method of the heart treatment apparatus of FIG. 1.

FIG. 4 illustrates another modified example of the electrode layout method of the heart treatment apparatus of FIG. 1.

FIG. 5 illustrates yet another modified example of the electrode layout method of the heart treatment apparatus of FIG. 1.

FIG. 6 illustrates still another modified example of the electrode layout method of the heart treatment apparatus of FIG. 1.

### DETAILED DESCRIPTION OF THE INVENTION

Hereunder is a description of one embodiment of the present invention, with reference to drawings.

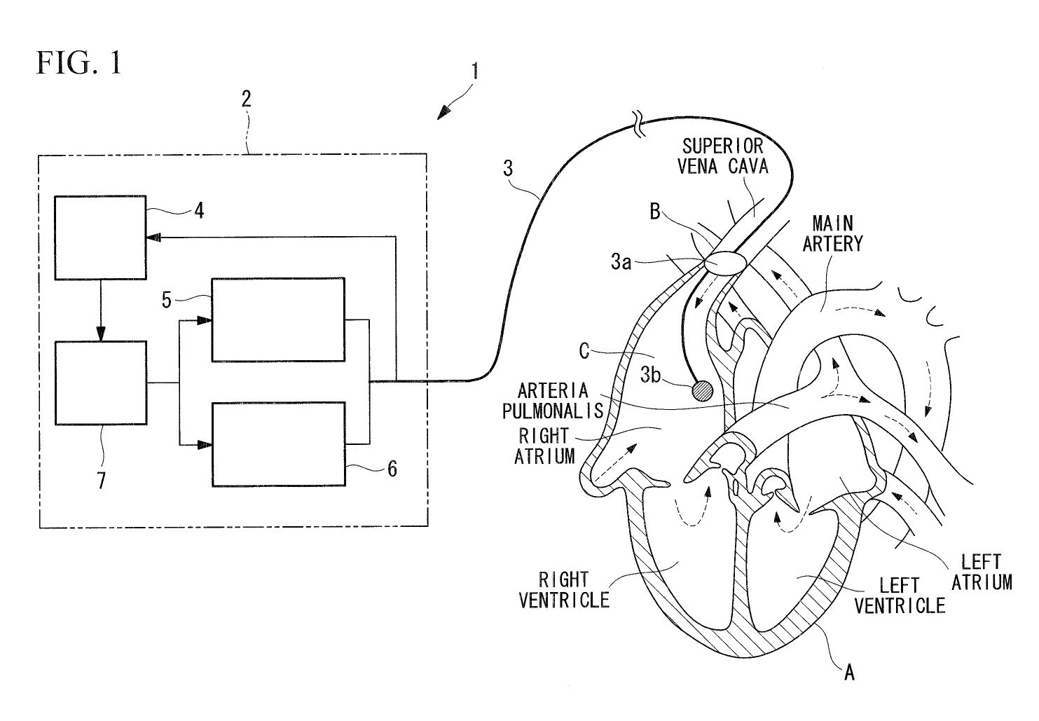

As shown in FIG. 1, in the electrode layout method of a heart treatment apparatus 1 according to this embodiment, a lead 3 which is connected to a main body 2 such as a pacemaker to be implanted in a body, is inserted in the heart A via the vena cava so as to arrange a venous electrode (electrode) 3a in the superior vena cava B and a heart electrode (electrode) 3b in the right atrium C.

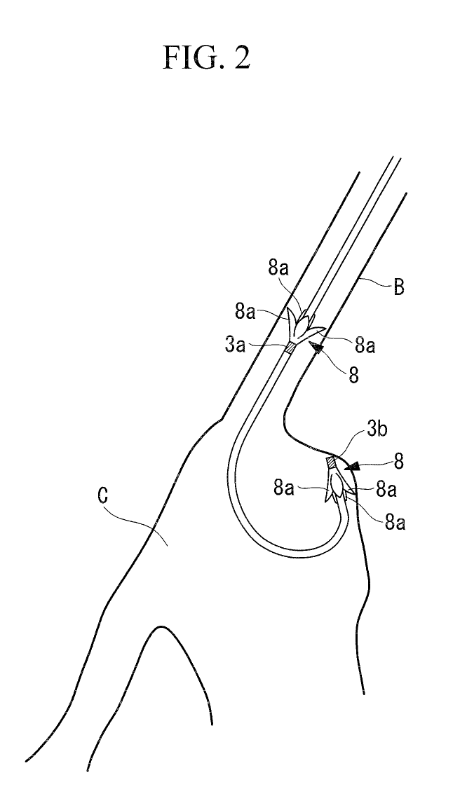

The venous electrode 3a and the heart electrode 3b are bipolar electrodes having mutually opposite polarities. The venous electrode 3a is fixed to the inner wall of the superior vena cava B. The heart electrode 3b is fixed to the inner wall of the right atrium C. The method for fixing the respective electrodes 3a and 3b is not specifically limited, and a method shown in FIG. 2 is employed.

FIG. 2 illustrates an example of a method for fixing the respective electrodes 3a and 3b to the inner wall of the superior vena cava B or the inner wall of the right atrium C. The lead 3 comprises, in the vicinities of the respective electrodes 3a and 3b, engaging portions 8 which have pluralities of blades 8a made of an electrically conductive material and arranged along the circumferential direction. One ends of the blades 8a on the distal side of the lead 3 are fixed to the lateral face of the lead 3 and the other ends of the blades 8a on the proximal side of the lead 3 are mutually separated in radial directions of the lead 3.

The engaging portion 8 is spread by changing its shape from an approximately cylindrical form along the lateral face of the lead 3 to an approximately pyramidical form towards the proximal end of the lead 3, so as to thereby effect engagement with the inner wall of the superior vena cava B or the right atrium C in the direction towards the proximal end of the lead 3. By so doing, the position of each electrode 3a or 3b is fixed to the superior vena cava B or the right atrium C, and each pulse voltage which has been output from each electrode 3a or 3b can be transmitted to the superior vena cava B or the right atrium C.

In the main body 2 are provided a heart rate meter 4 for measuring the heart rate, a pacing pulse supplier 5 for supplying a pacing pulse voltage between the venous electrode 3a and the heart electrode 3b, a stimulation pulse supplier 6 for supplying a stimulation pulse voltage therebetween, and a controller 7 for controlling the outputs of the pacing pulse voltage and the stimulation pulse voltage according to the condition of the heart A.

The heart rate meter 4 detects the potential of the heart electrode 3b relative to the potential of a reference electrode (not shown) provided on the proximal side of the lead 3. By so doing, the heart rate meter 4 obtains a change in the potential which fluctuates depending on the electrical activity of the heart A, that is to say, an electrocardiographic signal. Based on the waveform of the obtained electrocardiographic signal, the heart rate meter 4 measures the heart rate by using, for example, time intervals between timings when the magnitude, or the rate of change, of the potential of the electrocardiographic signal excels a predetermined threshold.

When the heart A falls in a bradycardia condition and the heart rate decreases, the pacing pulse supplier 5 supplies a pacing pulse voltage having a relatively high energy between the venous electrode 3a and the heart electrode 3b. By so doing, the right atrium C is stimulated to increase the heart rate.

When the heart A falls in a tachycardia or fibrillation condition and the heart rate increases, the stimulation pulse supplier 6 generates a stimulation pulse voltage having a sufficiently lower energy than the pacing pulse voltage and supplies it between the venous electrode 3a and the heart electrode 3b. By so doing, the vagus nerve which passes in the vicinity of the superior vena cava B is stimulated to decrease the heart rate.

As for the stimulation pulse voltage, for example, there is used a pulse voltage having an approximately equal voltage to the pacing pulse voltage and a sufficiently shorter pulse width than that of the pacing pulse voltage. Specifically, if the pacing pulse voltage has a pulse width of 0.5 to a few milliseconds, there is used a stimulation pulse voltage having a pulse width of 0.1 or shorter milliseconds.

The controller 7 monitors the heart rate measured by the heart rate meter 4 at all times. When the heart rate decreases below a predetermined lower limit, the controller 7 makes the pacing pulse supplier 5 generate the pacing pulse voltage to stimulate the right atrium C. At this time, the controller 7 controls the pacing pulse supplier 5 based on the electrocardiographic signal detected by the heart rate meter 4 so that the pacing pulse voltage can be supplied to the right atrium C at a timing out of the refractory period during which the heart A is incapable of responding to the pacing pulse voltage.

On the other hand, when the heart rate measured by the heart rate meter 4 increases above a predetermined upper limit, the controller 7 makes the stimulation pulse supplier 6 generate the stimulation pulse voltage to stimulate the vagus nerve. The stimulation pulse voltage is supplied between the both electrodes 3a and 3b at an arbitrary timing.

Hereunder is a description of the operation of the heart treatment apparatus 1 of which the venous electrode 3a and the heart electrode 3b are arranged in such a manner.

The heart treatment apparatus 1 according to this embodiment monitors the heart rate at all times. When bradycardia occurs in the heart A, the heart treatment apparatus 1 supplies the right atrium C with the pacing pulse voltage to increase the heart rate to a normal range. Moreover, when tachycardia or fibrillation occurs in the heart A, the heart treatment apparatus 1 supplies the vagus nerve with the stimulation pulse voltage via the superior vena cava B to decrease the heart rate to a normal range.

In this manner, according to the electrode layout method of the heart treatment apparatus 1 of this embodiment, the venous electrode 3a is arranged in the superior vena cava B which is communicated to the interior of the right atrium C, so as to stimulate the vagus nerve from the interior of the superior vena cava B. By so doing, leads which have been so far needed to be respectively arranged in the heart A and the vagus nerve, can be tailored in one common line of the lead 3 which is inserted in the right atrium C from the vena cava. Thus, advantageously, the setting of the lead 3 in the body can be simplified.

Furthermore, the heart electrode 3b is arranged in the right atrium C, by which the distance between the venous electrode 3a and the heart electrode 3b can be shortened. By so doing, these electrodes 3a and 3b can be used as bipolar electrodes to locally supply the pacing pulse voltage and the stimulation pulse voltage. Thus, advantageously, influences of these pulse voltages to the surrounding tissue can be diminished.

In addition, the electrodes 3a and 3b used for stimulating the heart A and the vagus nerve are commonly used to reduce the number of electrodes, by which the numbers of wirings and connectors connected from the respective electrodes 3a and 3b to the pacing pulse supplier 5 and the stimulation pulse supplier 6 can be reduced. Therefore, advantageously, the heart treatment apparatus 1 can be miniaturized. Moreover, even if the pacing pulse voltage and the stimulation pulse voltage were supplied to a same site in this manner, since the heart A and the vagus nerve show responses to pulse voltages having sufficiently different energies, the heart A and the vagus nerve can be selectively stimulated by the respective pulse voltages.

In the above embodiment, the heart electrode 3b is arranged in the right atrium C. However, instead of this arrangement, the heart electrode 3b may also be arranged in the superior vena cava B in the vicinity of the right atrium C. This arrangement also allows both stimulations to the right atrium C and the vagus nerve while detecting the electrocardiographic signal with the heart electrode 3b.

Moreover, in the above embodiment, both the vagus nerve and the heart A are stimulated by a set of the bipolar electrodes 3a and 3b. However, instead of this arrangement, sets of bipolar electrodes may also be respectively arranged in the superior vena cava B and the right atrium C. By so doing, the superior vena cava B and the right atrium C can be respectively locally stimulated.

Furthermore, in the above embodiment, the arrangement of the heart electrode 3b is not specifically limited, and the position of the heart electrode 3b can be selected according to the symptom of the patient in which the heart treatment apparatus 1 is to be installed.

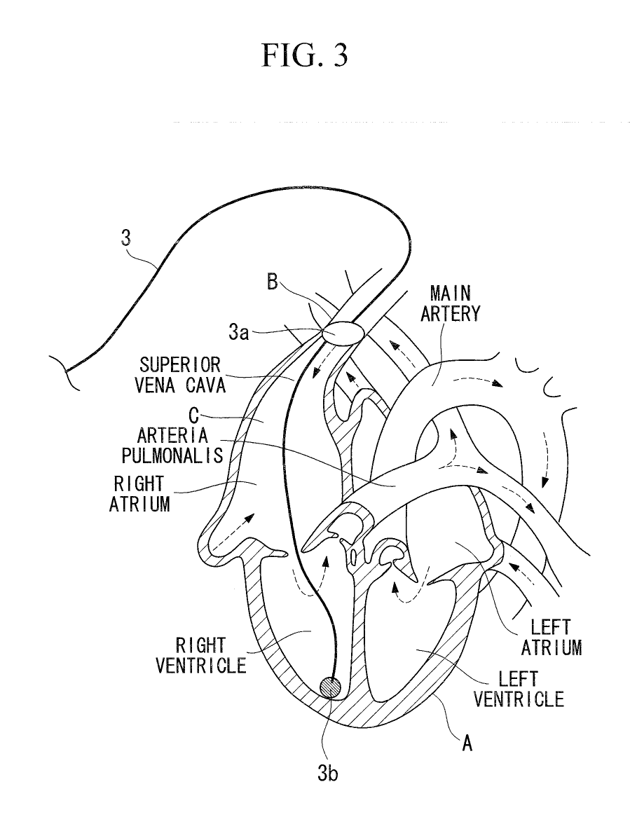

FIG. 3 illustrates an example of a case where the heart electrode 3b is arranged in the right ventricle. In this case, the pacing pulse voltage is supplied to the heart electrode 3b to stimulate the right ventricle while the stimulation pulse voltage is supplied to the venous electrode 3a to stimulate the vagus nerve. The venous electrode 3a and the heart electrode 3b may be respectively either monopolar or bipolar.

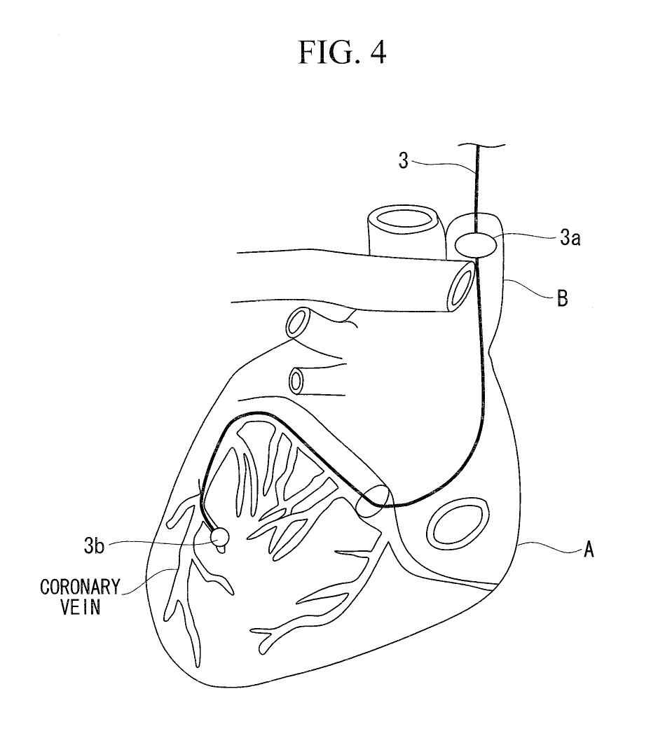

FIG. 4 illustrates an example of a case where the heart electrode 3b is arranged in the left ventricle. In this case, the lead 3 is inserted from the interior of the vena cava into the coronary vein on the outer face of the left ventricle, via the right atrium C. In this case, the heart A and the vagus nerve are stimulated, as well as the case where the heart electrode 3b is arranged in the right ventricle. The venous electrode 3a and the heart electrode 3b may be respectively either monopolar or bipolar.

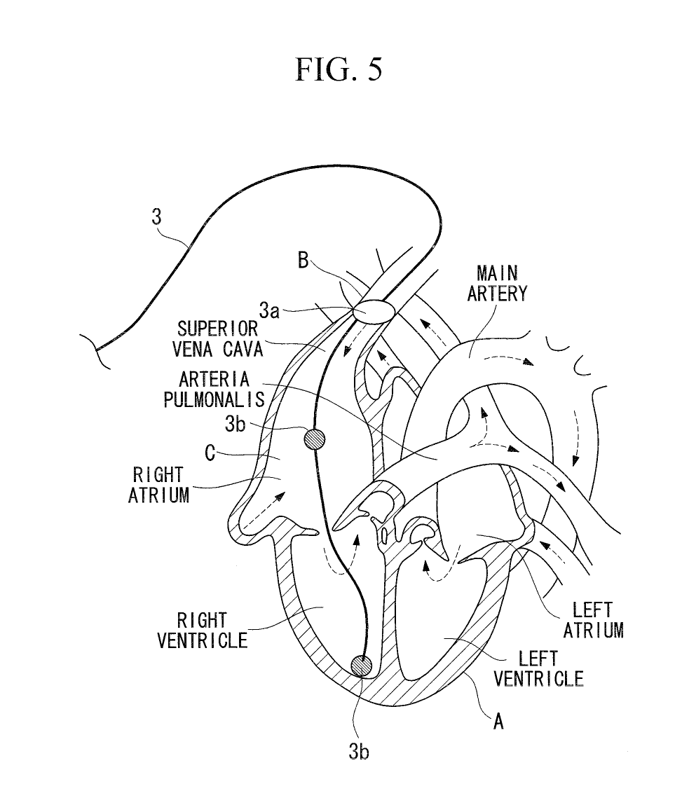

FIG. 5 illustrates an example of a case where two of the heart electrodes 3b are arranged in both the right atrium C and the right ventricle. In this case, the heart rate is measured in both the right atrium C and the right ventricle, and the controller 7 supplies either one, or both of, the right atrium C and the right ventricle with the pacing pulse voltage, based on the heart rate measured in the right atrium C and the right ventricle. The venous electrode 3a and the heart electrode 3b may be respectively either monopolar or bipolar.

In addition, in the above embodiment, the heart treatment apparatus 1 supplies the pacing pulse voltage and the stimulation pulse voltage by different configurations. However, instead of this, these pulse voltages may also be supplied by a common configuration.

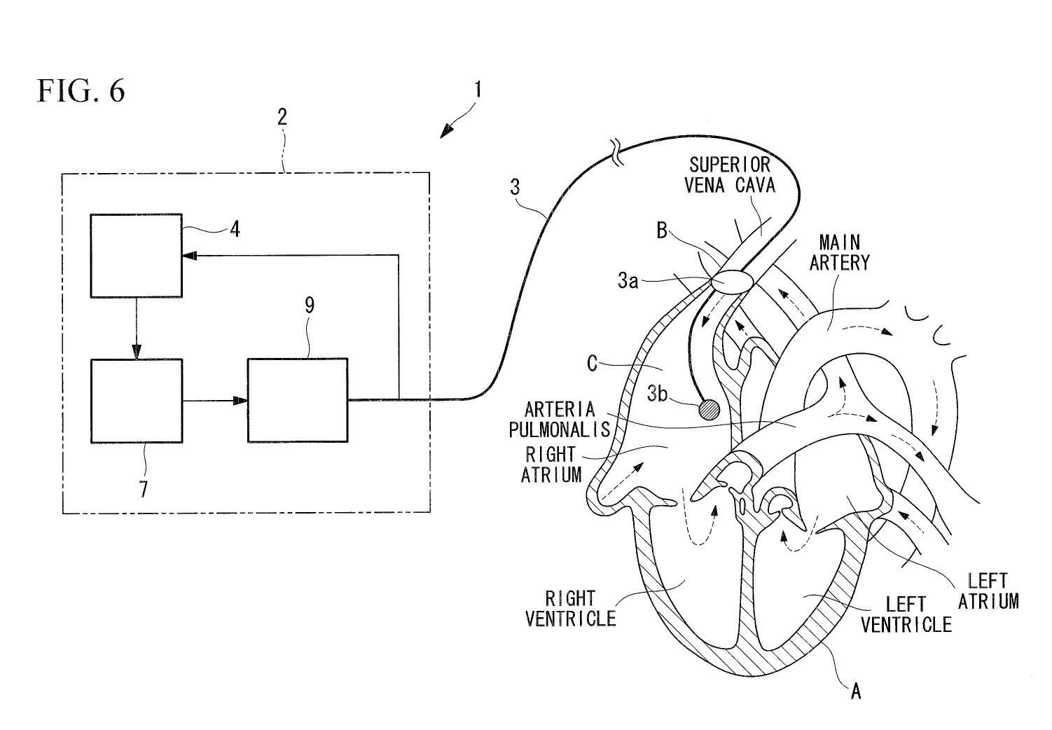

FIG. 6 illustrates an example of a case where the pacing pulse voltage and the stimulation pulse voltage are supplied by a pulse supplier 9 with use of the setup of the lead 3 in FIG. 1. The pulse supplier 9 comprises, for example, a capacitor (not shown) connected to the lead 3, to generate the pacing pulse voltage or the stimulation pulse voltage by elongating or shortening the discharge time for discharging electricity from the capacitor to the lead 3.

The present invention has the following aspect.

The present invention provides an electrode layout method of a heart treatment apparatus, comprising: upon installation of the heart treatment apparatus comprising an apparatus main body and a lead extending from the apparatus main body, arranging at least one electrode out of a plurality of electrodes provided on the lead in the superior vena cava.

According to the present invention, the stimulation pulse voltage for stimulating the vagus nerve is supplied from the apparatus main body to the electrode arranged in the superior vena cava via the lead, by which the vagus nerve existing in the vicinity of the superior vena cava can be stimulated. In addition, another electrode may also be arranged in, or in the vicinity of, the heart which is adjacently communicated to the superior vena cava. With this arrangement, the heart can be stimulated by supplying the pacing pulse voltage for pacing the heartbeat, from the apparatus main body to the another electrode via the lead.

In this way, the vagus nerve is stimulated from the superior vena cava which is communicated to the heart. Therefore, the electrode for stimulating the heart is provided on the same lead where the electrode for stimulating the superior vena cava is provided. Thus, leads which have been so far respectively arranged in the heart and the vagus nerve, can be tailored in one common line of lead. By so doing, the lead setting can be simplified while allowing both heart stimulation and vagus nerve stimulation so as to thereby shorten the operation time required for implanting the heart treatment apparatus.

In the above method, two electrodes provided on the lead may also be spacedly arranged in the superior vena cava.

By so doing, the pacing pulse voltage and the stimulation pulse voltage can be supplied between common electrodes arranged in the superior vena cava, because of which the number of electrodes can be reduced while locally supplying the respective pulse voltages.

Moreover, in the above method, at least one electrode out of the other electrodes provided on the lead may be arranged in the right atrium, the right ventricle, or the left ventricle.

By so doing, each site of the heart can be more efficiently stimulated.

Furthermore, in the above method, two electrodes out of the other electrodes provided on the lead may also be spacedly arranged in the right atrium, the right ventricle, or the left ventricle.

By so doing, each site of the heart can be locally supplied with the pacing pulse voltage.

Also, in the above method, other electrodes provided on the lead may be arranged in the right atrium and the right ventricle at least one by one.

By so doing, both the right atrium and the right ventricle can be stimulated.

In addition, in the above method, other electrodes provided on the lead may also be spacedly arranged in the right atrium and the right ventricle two by two.

By so doing, the right atrium and the right ventricle can be locally supplied with the pacing pulse voltage.

What is claimed is:

1. An electrode layout method of a heart treatment apparatus, comprising: upon installation of the heart treatment apparatus comprising an apparatus main body and a lead extending from the apparatus main body, arranging at least one electrode out of a plurality of electrodes provided on the lead in the superior vena cava.

2. An electrode layout method of a heart treatment apparatus according to claim 1, wherein two electrodes provided on said lead are spacedly arranged in said superior vena cava.

3. An electrode layout method of a heart treatment apparatus according to claim 1, wherein at least one electrode out of the other electrodes provided on said lead is arranged in the right atrium.

4. An electrode layout method of a heart treatment apparatus according to claim 3, wherein two electrodes out of the other electrodes provided on said lead are spacedly arranged in said right atrium.

5. An electrode layout method of a heart treatment apparatus according to claim 1, wherein at least one electrode out of the other electrodes provided on said lead is arranged in the right ventricle.

6. An electrode layout method of a heart treatment apparatus according to claim 5, wherein two electrodes out of the other electrodes provided on said lead are spacedly arranged in said right ventricle.

7. An electrode layout method of a heart treatment apparatus according to claim 1, wherein at least one electrode out of the other electrodes provided on said lead is arranged in the left ventricle.

8. An electrode layout method of a heart treatment apparatus according to claim 7, wherein two electrodes out of the other electrodes provided on said lead are spacedly arranged in said left ventricle.

9. An electrode layout method of a heart treatment apparatus according to claim 1, wherein other electrodes provided on said lead are arranged in the right atrium and the right ventricle at least one by one.

10. An electrode layout method of a heart treatment apparatus according to claim 9, wherein other electrodes provided on said lead are arranged in said right atrium and said right ventricle two by two.
